(19) United States
(12) Patent Application Publication
(10) Pub. No.: US 2010/0300459 A1
(43) Pub. Date: Dec. 2, 2010

(54) DRAPE AND METHOD OF USING SAME

(76) Inventor: Anthony C. Lair, Alpharetta, GA (US)

Correspondence Address: NORA M. TOCUPS, P.O BOX 698, 140 PINECREST AVE, DECATUR, GA, 30030, US

(21) Appl. No.: 12/788,257

(22) Filed: May 26, 2010

Related U.S. Application Data
(60) Provisional application No. 61/181,016, filed on May 26, 2009.

(51) Int. Cl. A61B 19/08

(52) U.S. Cl. 128/853; 128/855; 206/440

(57) ABSTRACT

A drape or wrap, kit, and methods of use thereof. The drape may be used with an infant. The drape may be a sheet of biocompatible medical grade plastic such as polyethylene. The sheet may be substantially: sterile, without static, transparent, non-abrasive to skin of the infant, and fluid-impermeable. The sheet may allow for folding over or around the infant, or to conform to body contours. The sheet may allow for heat from an external source to penetrate to the infant. The sheet may include a fenestration, and may include a perforation from the fenestration to the sheet's edge. An adhesive strip on the sheet may be used to secure it. A kit may include a package enclosing a sterilized folded wrap. The package may be opened to allow removal of the wrap. The wrap may be unfolded to a size to cover all but the head of the infant.

### CROSS-REFERENCE TO RELATED APPLICATION

The present application claims priority to and benefit of the prior filed co-pending and commonly owned provisional application, filed in the United States Patent and Trademark Office on May 26, 2009, assigned Ser. No. 61/181,016, entitled Drape and Method of Use Thereof and incorporated herein by reference.

### FIELD OF THE INVENTIONS

The inventions relate generally to medical devices and methods, and particularly relate to medical devices and methods as may be used with infants.

### BACKGROUND

Hypothermia is considered by the World Health Organization to be a “top killer” during the neonatal period of an infant. Even for full-term babies, cold stress has been associated with many problems including lethary, hypotonia, poor feeding, weight loss, abdominal distention, vomiting, restlessness, pallor, cool skin, tachypnea, respiratory distress and reduced core temperature. Reductions in body temperature may delay transition from fetal to neonatal circulation. Thus, hypothermia, cold stress and reductions in body temperature are to be avoided.

### SUMMARY

Generally stated, the inventions relate to drapes or wraps that may be used with infants, and methods of us thereof. The inventions are described herein as being used with specific elements and features, but should not be limited to the particular examples given. One or more of the inventions may be used in other circumstances and/or with other elements or features.

The invention relates to a “drape” (also referred to as “wrap”). An exemplary embodiment of the invention may be used as or as part of a sterile barrier in creating and maintaining a sterile field when a procedure is conducted on a patient. An even more important feature of the invention may be that it may reduce the risk of cold stress when it is used—especially with premature and very low birth weight infants. The invention may be embodied in a polyethylene wrap designed to reduce the risk of cold stress post-delivery and during sterile procedures. To enhance patient safety, the wrap may be made from biocompatible medical grade plastic, packaged sterile and without dangerous static. The exemplary wrap may have one or more of the following features and offer the following advantages:

1. Clear visualization of the patient during sterile procedures

2. Does not impede radiant heat

3. Sterile

4. Low cost

5. Fenestrated

6. Perforated

7. Sterile packaging without static electricity

A first embodiment of the invention relates to a drape or wrap that may be used with an infant. The drape may be a sheet made of biocompatible medical grade plastic such as polypropylene. The sheet may be at least substantially: sterile, without static, transparent, and/or fluid-impermeable. The sheet may be flexible enough to allow for being folded over or around the infant. The sheet may be non-abrasive to skin of the infant. Advantageously, the sheet may allow for heat such as radiated heat from an external source to penetrate through the sheet to the infant.

Another embodiment of the invention may include one or more characteristics of the drape mentioned in the immediately preceding paragraph. In addition, this other embodiment may include a fenestration. A perforation or a line of perforations may connect the fenestration to an edge of the sheet or drape.

The invention also allows for another embodiment relating to a drape for use with an infant. The drape may include a polyethylene plastic sterile sheet sized large enough at least to substantially cover all but the head of the infant so as to create a substantially sterile field between the infant and the sheet. The sheet may have a fenestration. The sheet also may have at least a perforation between the fenestration and an edge of the sheet. The sheet of this embodiment may be substantially: transparent, 2.0 millimeters in thickness, without static; non-abrasive to the skin of an infant; and/or fluid-impermeable. The sheet may allow heat to penetrate. The sheet may be flexible enough to fold around an infant or to mold to conform to the body contours of an infant. This embodiment also may include a means to secure the sheet of the drape to an element such as a support for the infant. For example, the sheet may have at least an adhesive strip to use as the securing means.

The invention also may be embodied in a kit or kits. An exemplary kit according to the invention may be used in conserving body temperature of an infant. The kit may include a package enclosing a folded polyethylene wrap. The wrap may be sterilized in the package. The package may be openable to allow selective removal of the folded wrap from the package. The wrap may be selectively unfoldable to a size at least to substantially cover all but the head of an infant. The wrap may be flexible enough to fold around the infant. The wrap may be transparent. The wrap may be non-abrasive with respect to skin of the infant. Also, the wrap may be substantially fluid-impermeable.

Exemplary embodiments according to the invention have been summarized above. Many more are possible; the inventions are not to be limited to these examples. Other features and advantages of the inventions may be more clearly understood and appreciated from a review of the following detailed description and by reference to the appended drawings and claims.

### BRIEF DESCRIPTION OF THE DRAWINGS

FIG. 1 is a drawing of an exemplary embodiment of the invention.

FIG. 2 is a drawing of another exemplary embodiment of the invention as it may be used with an infant.

FIGS. 3A-3D are drawings of yet another exemplary embodiment of the invention as it may be used with an infant.

FIG. 4 depicts an exemplary kit according to the invention.

### DETAILED DESCRIPTION

The inventions are described herein with reference to exemplary embodiments, alternative embodiments, and also with reference to the attached drawings. The invention, however, can be embodied in many different forms and carried out in a variety of ways, and should not be construed as limited to the embodiments set forth in this description and/or the drawings. The exemplary embodiments that are described and shown herein are only some of the ways to implement the inventions. Elements and/or actions of the inventions may be assembled, connected, configured, and/or taken in an order different in whole or in part from the descriptions herein.

Generally stated, the invention relates to the care of infants, and in particular, to infants who may be born premature or with a very low birth weight (V.L.B.W.). An embodiment of the invention may be used to keep such infants from losing critical body temperature. Other embodiments of the invention may keep an infant from losing critical body temperature and also provide a sterile field for procedures that may need to be carried out on the infant. Yet other embodiments may provide a kit that includes a folded drape or wrap in sterile packaging that allows for easy removal of the drape and application to the infant as needed.

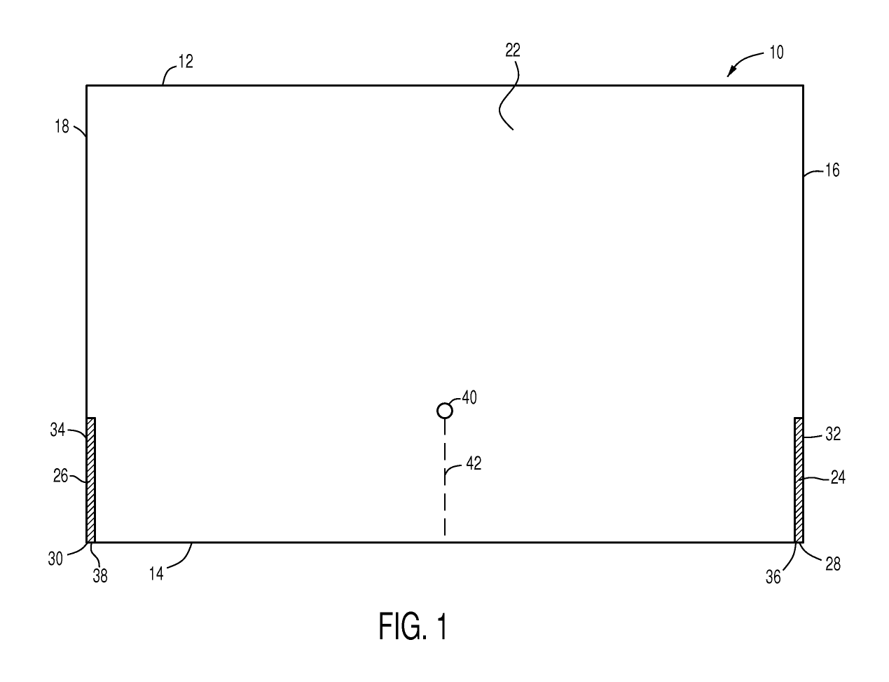

FIG. 1 illustrates an exemplary embodiment of a drape 10. The exemplary drape 10 is substantially rectangular, but may be otherwise shaped. In the orientation shown in FIG. 1, the drape 10 is wider than it is tall. The longer sides 12, 14 are about 39 inches. The shorter sides 16, 18 are about 31.5 inches. Other embodiments may be of respectively different sizes and/or shapes. For example, another embodiment provides a size of about 32 inches by 50 inches. Generally, a drape according to this embodiment 10 may be made long and wide enough to create a sterile field when in use with an infant. In this example, the exemplary drape 10 may be sized for use with an infant (preemie, neonate). Other sizes may be used in other embodiments.

The exemplary drape 10 is substantially clear or transparent, a characteristic that allows a user to maintain visualization of the patient as may be required by certain medical guidelines. (ref NANN PICC Guidelines Pg.17 step 25) Other drape embodiments may be opaque in whole or in part, and/or may have color in whole or in part. An advantage of the substantially clear drape 10 is that a person may see through the drape 10. For example, a doctor carrying out a procedure on an infant draped in drape 10 has clear visualization of the infant during sterile procedures. As another example, the clear drape 10 allows a clinician to view the patient as necessary so as to be able to respond rapidly to clinical issues. Another advantage of the exemplary drape 10 is that it may be made to cover a patient such as infant as much as possible (for heat and sterile reasons), but the exemplary drape 10 also allows for substantially full observation of the infant because they are transparent.

The exemplary drape 10 is made substantially completely of biocompatible medical grade polyethylene plastic. The use of plastic in the exemplary drape 10 contrasts with the paper drapes previously used. A common problem in the use of paper drapes is that they may impede radiant heat. The plastic of the exemplary embodiment 10 does not generally impede radiant heat. The polyethylene drapes may permit an infant to gain heat through radiation and may also reduce the amount of evaporative or other heat and/or moisture loss. Thus, the plastic of the exemplary embodiment 10 may reduce the risk of cold stress post-delivery and during sterile procedures. The exemplary drape 10 complies with the guidelines for temperature control published by the 2005 National Resuscitation Program of the American Academy of Pediatrics.

Also being made of plastic, the exemplary drape 10 is generally fluid-impermeable. The exemplary drape 10 may be liquid repellant to prevent strike-through of liquids such as water, blood, and the like.

Another advantage of the use of polyethylene plastic is that it may make the wrap 10 relatively flexible and capable of being draped for ease of use. The thinness of the plastic used may contribute to this advantage. An exemplary embodiment of the invention such as drape 10 may have a thickness of about 20 millimeters throughout. This thickness of plastic may aid in maintaining a baby's body temperature during a procedure while still allowing for ease of use. Another embodiment may have another thickness such as 1.5 millimeters Other embodiments may have different thicknesses in whole or in part.

As noted, the exemplary drape 10 is made substantially completely of biocompatible medical grade polyethylene plastic. Other embodiments may vary wholly or in part in composition. For example, an alternative embodiment may have a polyethylene plastic central area with a paper (and/or other material) border.

For ease of reference and given the orientation of the drawing of the exemplary drape 10 in FIG. 1, the upper long side 12 of exemplary drape 10 is referred to herein as the “upper side 12” and its substantially parallel lower long side 14 is referred to herein as the “lower side 14”. The right shorter side 16 of the drape 10 is referred to herein as the “right side 16” and its substantially parallel opposite side 18 is referred to herein as the “left side 18”. Given the relative thinness of the plastic, the drape 10 is substantially essentially two-sided. The side 22 of the drape 10 “closest” to the viewer is referred to herein as the “top side 22”, and its opposite side (not visible in FIG. 1) is referred to herein as the “bottom side”.

Another advantage of the exemplary drape 10 shown in FIG. 1 is that it may be secured. For example, the drape 10 may be secured to a procedure table or to the dropsides of a bassinet holding the infant. Securing the drape 10 may aid in preventing a breach of the sterile field created by the drape 10 should the baby move or the drape 10 otherwise be dislodged from its original position in creating the sterile field. The drape 10 may be secured in any appropriate manner.

In the embodiment shown in FIG. 1, adhesive areas are provided on the drape 10 for use in securing the drape 10. The adhesive areas are covered by tape strips until the drape 10 is to be secured. More particularly described, the substantially rectangular strips 24, 26 are located on the drape 10 and positioned with respect to the lower corners 28, 30 of the drape 10. Generally stated, the strips 24, 26 cover respective adhesive areas. One or both strips 24, 26 may be removed to uncover the adhesive areas. Thus, the strips 24, 26 also may be referred to as removably adhered protective covering release strips.

In this embodiment, each strip 24, 26 is approximately six (6) inches long and 1.25 inches wide. Other embodiments may have strips that vary in size and shape. For example, another embodiment may have strips that are four (4) inches long. Each strip 24, 26 is opaque and white in color. The opacity and the white color may allow for quick identification of the strips 24, 26 as compared to the clear remainder of the drape 10. The quick identification of the strips 24, 26 may facilitate the use of the drape 10. Other embodiments may have strips of different sizes and shapes (in whole or in part), and/or may be partially or completely of another color or transparency.

The outer long sides 32, 34 of each strip 24, 26 are generally parallel respectively to the right and left sides 16, 18 of the drape 10. In this embodiment, the outer long sides 32, 34 of the strips 24, 26 are substantially even respectively with the right and left sides 16, 18 of the drape 10. In other words, the edges of the outer long sides 32, 34 match up substantially with the bottom edges of the right and left sides 16, 18.

Similarly, the outer short sides 36, 38 of each strip 24, 26 are generally parallel respectively to the outer edges of the lower side 14. In this embodiment, the outer short sides 36, 38 of the strips 24, 26 are substantially even respectively with the right and left outer edges of the lower side 14. In other words, the edges of the outer short sides 36, 38 match up substantially with the outer edges of the lower side 14. In effect, the strips 24, 26 are placed substantially in the bottom corners 28, 30 of the drape 10 with the outer long sides 32, 34 of the strips 24, 26 matching up respectively with the lower parts of the right and left sides 16, 18 of the drape 10. Other embodiments may vary in placement (in whole or in part).

The strips 24, 26 are removable. Each of the strips 24, 26 may cover an adhesive that is affixed to the top side 22 of the drape 10 under the substantially whole of the strips 24, 26. In other words, the strips 24, 26 are positioned to protect the adhesive from adhering prior to the determined use of the adhesive. The areas of the top side 22 of the drape 10 that include the adhesive are referred to herein as “adhesive areas”. In this embodiment, the adhesives areas are those areas underneath the strips 24, 26 and on the top side 22 of the drape 10. Other embodiments may have adhesive positioned differently (in whole or in part) and/or implemented in other ways.

The adhesive may be of any kind appropriate to use its in sterile environments and/or other requirements and/or recommendations. Any of the adhesives well known in the art for this purpose may be used. The adhesive may be non-toxic, stable to ordinary sterilization procedures, and removably adherable to the surface to which it will be applied. The adhesive areas are designed to allow for the drape 10 generally to be secured to the rails of the bed or bed surface, (or other element) and not to stick to the patient. For example, the adhesive areas may be used to secure the drape 10 during a procedure.

The exemplary drape 10 includes adhesive areas that are covered by adhesive strips 24, 26. Other embodiments, however, may be differently configured. For example, an embodiment may include a wrap without any adhesive areas. As another example, a wrap may have only one adhesive area and in a place different than in the exemplary embodiment 10. Further, another embodiment may have more than two adhesive areas placed in areas other than the adhesive areas in the exemplary drape 10. In addition, an exemplary embodiment may have one or more adhesive areas (in the same place as exemplary embodiment 10 or not), and one of more of the adhesive areas may be protected prior to use in some way other than by an adhesive strip. Other embodiments may have ways or means of securing a drape different from the use of removable tape covered adhesive. Other ways of securing may include Velcro® (hook/eye closure), ties, snaps, etc.

The exemplary drape 10 is fenestrated. That is, the exemplary drape 10 includes an opening 40 in the form of a substantially round hole of approximately 1⅜^{th} inches in diameter. The opening 40 is located in this embodiment 10 approximately 7¾^{th} inches from the lower side 14 of the drape 10. In addition, the opening 40 is generally centered between the right and left sides 16, 18 of the drape 10. Thus, the opening 40 is located at approximately 19.5 inches from either the right or left side. Other embodiments may have more than one opening, and/or may have an opening(s) of different sizes, shapes and other characteristics.

The opening 40 in the drape 10 provides many advantages. When the drape 10 is positioned over a patient, the opening 40 may reveal a small part of the patient, but otherwise the patient may be covered by the drape 10 forming a sterile barrier yet preventing heat loss. The practitioner may carry out a procedure on the patient through the opening 40 in the drape 10. The opening 40 may be selectively enlarged as needed for a procedure.

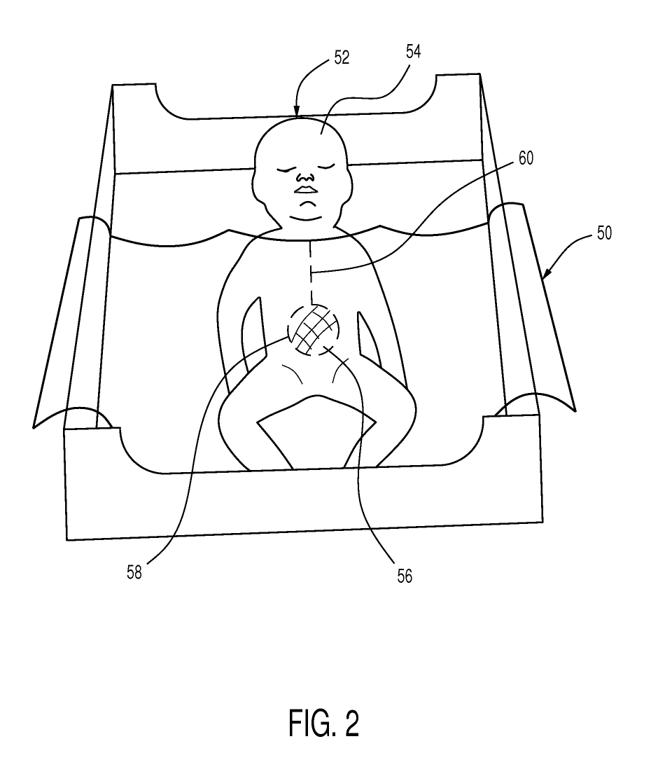

FIG. 2 illustrates an exemplary use of another exemplary drape 50 with a young patient 52. The clear drape 50 is placed so that it covers all of the patient 52 but his or her head 54 and an area 56 of the patient's body beneath the fenestration 58 in the drape 50. A practitioner may carryout a procedure on the exposed area 56 of the patient 52 through the opening 58 in the drape 50.

The inclusion of the opening 40 in the drape 10 may allow for the use of only a single drape (rather than many) over the patient during a procedure. The same pertains to the exemplary drape 50 shown in FIG. 2. For example, assume a part of a patient needs to be revealed so the practitioner may carry out a procedure. If only drapes without openings are available, the patient may have to be covered with more than one drape so the appropriate part of the patient is revealed but other parts are covered for a sterile barrier. If the patient is small in size, such as an infant, the patient may be overloaded by all these extra drapes. A single drape with an opening allows the appropriate part of the infant to be revealed for the procedure allowing for easy access to the surgery site without the need of manually creating an access point or hole in a plastic drape. Advantageously, the fenestration comes pre-cut in the exemplary embodiments having such fenestrations. A pre-cut fenestration may allow for ready and easy placement of the fenestration over the surgical site and also allows for easy removal of the drape from the patient at the end of a procedure.

Another time a patient may be draped is when there is a dressing change such as a change of a dressing for a peripherally inserted central catheter (PICC). The dressing change is recommended to be made using sterile technique such as the use of a surgical drape. A fenestrated drape such as provided by the exemplary embodiments 10, 50 may be used to delineate the area of the dressing but otherwise cover the infant as much as possible (for heat and sterile reasons). The insertion of a umbilical vessel catheter is another occasion where a fenestrated drape may prove advantageous for multiple reasons.

An advantage of the fenestration may be that it allows the exemplary drapes 10, 50 to be positioned so as to avoid covering (any or all) of the head of the patient. For example, the drape may be positioned so the fenestration is placed at the insertion site of the PICC (and/or other location of procedure) with the long sides of the drape being perpendicular to the body of the patient and the short sides being generally parallel to the length of the body of the patient.

The exemplary drapes 10, 50 include a single fenestration. Other embodiments may be differently configured. For example, an embodiment may lack a fenestration. Another embodiment may have a fenestration in a different location and/or of a different size and/or shape. Further, another embodiment may have more than one fenestration.

The exemplary drape 10 also includes a line of perforation 42 (also referred to as “perforation”) relative to the opening 40 in the drape 10. The perforation 42 extends from about center bottom of the opening 40 in a substantially straight line of approximately 7¾^{th} inches to the approximate center of the lower side 14. Thus, the line of perforation 42 is generally parallel to the lower parts of the right and left sides 16, 18 of the drape 10, and generally perpendicular to the lower side 14. Other positions, sizes and characteristics (including numbers of lines of perforation) may be included in other embodiments. For example, an embodiment may lack a perforation line, or include a perforation line positioned differently than in the exemplary embodiment 10. As another example, an embodiment may have more than one perforation line for each fenestration. If an embodiment has more than one fenestration, each may have one or more perforation lines or not.

The line of perforation 42 allows a practitioner to selectively tear apart the drape 10 along its line of perforation 42. Without the perforation, the drape 10 may be generally difficult to tear apart and the drape might be difficult to remove from around a catheter post insertion and/or other.

The line of perforation 42 in the exemplary drape 10 offers advantages. First, the line of perforation 42 facilitates the selective tearing apart of the drape 10. A practitioner may effectively enlarge the area of the opening 40 by tearing the line of perforation from the opening 40 towards the lower side 14. Enlarging the opening 40 may allow the practitioner to access additional parts of the patient than just revealed by the opening 40. The selective aspect of the line of perforation allows the practitioner to keep the parts of the patient revealed by the torn line covered until an appropriate time in the procedure. This selective aspect provides for keeping the sterile barrier as large and in place as much as possible.

Another advantage of the line of perforation 42 of the exemplary drape 10 is that all or part of the perforation 42 may be torn. For example, positioning of the drape 10 over a patient, especially a small patient, may require tearing the line of perforation 42 partially from the lower side 14 of the drape 10 so as to be better able to position the drape 10 into a sterile barrier around the patient.

Finally, an advantage of the line of perforation 42 may be that it allows for the relatively easy removal of the exemplary drape 10 when the drape is no longer needed such as at the end of a procedure. For example, assume a practitioner inserts a catheter into a patient by making use of the fenestration 40 mentioned above. Once the catheter is in place at the end of the procedure, the practitioner may tear the line of perforation 42 up to the opening 40 so as to relatively easily remove the drape 10 without effecting the catheter.

The exemplary embodiment 50 shown in FIG. 2 shows an illustrative perforation line 60 similar to that in embodiment 10. The comments made with respect to the line of perforation 42 in drape 10 may also apply to the perforation line 60 in embodiment 50.

FIGS. 1 and 2 illustrate exemplary embodiments 10, 50 of the invention that may keep an infant from losing critical body temperature and also provide a sterile field for procedures that may need to be carried out on the infant. The fenestrations and lines of perforation in these embodiments 10, 50 may aid practitioners in carrying out procedures on infants while maintaining the body temperatures of the infants. At times, however, an infant may simply need to be kept from losing body temperature. Such a time may be immediately post-delivery.

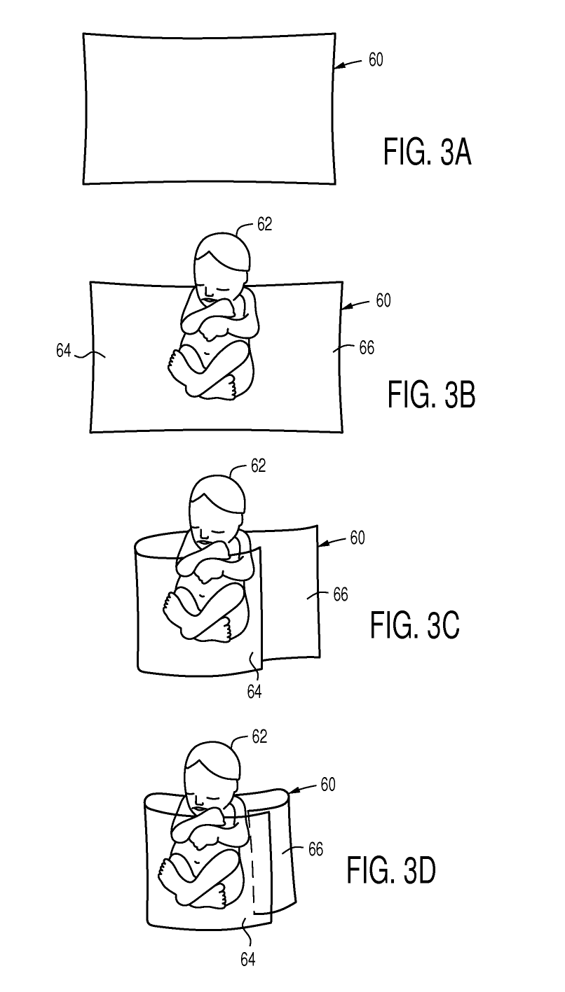

FIGS. 3A-3D illustrate the use of another exemplary embodiment 60 of the invention as it may be used in wrapping an infant 62. A drape or wrap that is used in such a way may be referred to as an occlusive drape or wrap. Particularly, FIG. 3A shows the exemplary wrap 60 by itself. Like the previous exemplary drapes 10, 50, this wrap 60 is generally rectangular with longer top and bottom than sides. Another embodiment may be square such as an embodiment being 24 inches square in size and shape. The exemplary wrap 60 is made of polyethylene, is clear throughout (transparent), and is sterile.

The exemplary wrap 60 has a thickness throughout of about 1.5 millimeters. The relatively thinness of the wrap 60 has advantages in that it allows for the wrap 60 to be relatively easily folded over a baby 62 as shown in FIGS. 3B-3D. In FIG. 3B, all but the head of the baby 62 is placed (back down) in the approximate center of the wrap 60. As shown in FIG. 3C, the left side 64 (looking at FIG. 3B) is folded over the top of the baby 62. The wrap 60, as noted, is transparent. Thus, the baby 62 may be seen through the folded over left side 64 of the wrap 60. In FIG. 3D, the right side 66 of the wrap 60 is folded as appropriate over the baby 62 to encompass the baby 62 completely within the wrap 60. Again, the wrap 60, as noted, is transparent. Thus, the baby 62 may be seen through the folded over left side 64 and right side 66 of the wrap 60. In FIG. 3D, part of the right side 66 of the wrap 60 appears to be positioned at least somewhat under the left side 64 of the wrap 60 to envelope the baby 62. Other means of wrapping are possible.

For example, the exemplary wrap 60 may be made to mold around the different contours of the baby 62 so that there is a closer fit of the wrap 60 around the baby 62 than shown in FIG. 3D. Advantageously, a practitioner may determine whether to “loosely” or “tightly” or something “in between” wrap a patient depending upon the circumstances.

Yet another advantage of the polyethylene material used in the exemplary embodiments of the invention is that it may be a relatively soft material (durometer) with respect to the skin of an infant. Babies generally have soft skin, and so it is important to use materials that come in contact with their skin that are at least non-abrasive to that skin. Very low birth weight (V.L.B.W.) babies may have especially delicate (very thin) skin. Thus, it is especially an advantage of exemplary embodiments of the invention to be of soft material so as not to injure such soft skin.

The advantages of the exemplary embodiments of the invention in maintaining a baby's body temperature have been discussed. Related to this feature is that the material used in the exemplary embodiments may allow radiant heat from an external source to penetrate through the material to warm the baby covered or wrapped by an exemplary drape. For example, a baby may be placed under a radiant warmer having a warming technology that adjusts to the patient's core temperature needs. Typically, an adult is not placed under a radiant warmer. If a baby is covered or wrapped in paper or towels, the paper or towels will impede the radiant heat from reaching the patient. In contrast, the material of the exemplary embodiments allows the radiant heat to reach the infant enclosed with the exemplary drape 10.

Drapes such as the exemplary drapes 10, 50, 60 may be individually packaged to be ready for use. A drape may be packaged in sterile packaging so the drape may be used for sterile procedures. Further, the packaging of the drape may be accomplished so as to eliminate (at least substantially) static electricity. Static electricity may be dangerous in a high oxygen environment where the drape may be used.

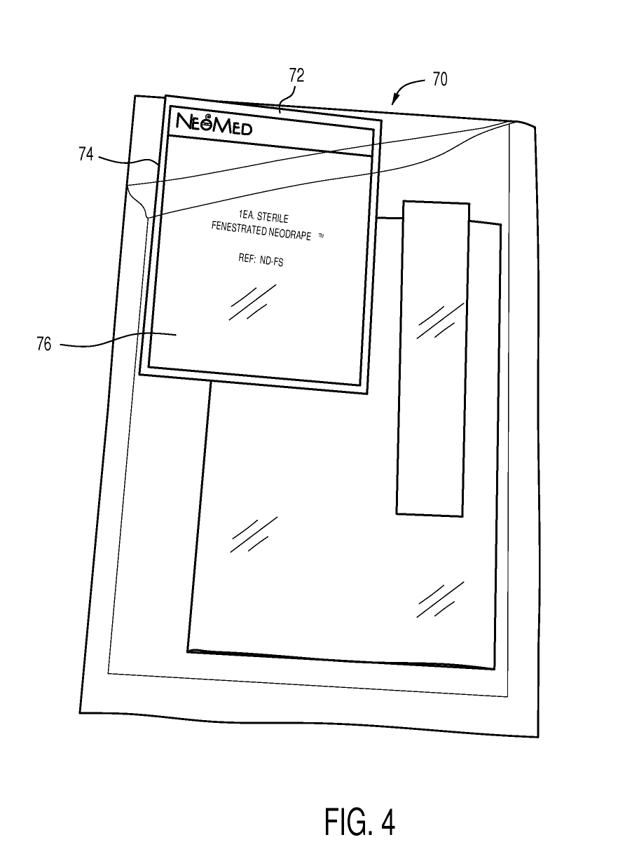

FIG. 4 illustrates an exemplary kit 70 including an exemplary drape 72 according to the invention partially inserted into a package 74. The top side of the package 74 (facing the viewer) is transparent so the drape 72 may be seen within. The bottom side of the package is opaque. The drape 72 may be folded into a compact unit which can be withdrawn and/or held in one hand. The drape 72 may be readily unfolded and relatively quickly and generally aseptically applied to the patient.

As noted, the folded drape 72 may be easily inserted into, and sealed within, a suitable container such as a plastic bag 74. The packaged folded drape may be sterilized with steam, ethylene oxide gas (ETO) sterilization, or radiation. Advantageously, the sterilized folded drape of the present invention may be quickly unfolded and easily applied to the patient.

Another advantage of the kit 70 shown in FIG. 4 is that it may include a label 76 such as shown in FIG. 4. The top side of the label 76 (facing the viewer) may include identification information about the drape 72 contained in the kit 70. The reverse side of the label (not shown in FIG. 7) may include instructions for use. In this example, the label's instructions for use give the following information:
NEODRAPE™ is a single use product. Do Not Reuse.
Do not use the NEODRAPE™ for infants born with blistering skin conditions that preclude the use of an occlusive wrap.
Do not overheat the NEODRAPE™
Do not place the NEODRAPE™ over the infant's face, ensure the infant's airway is clear at all times.
Do not wrap the infant too tightly in the NEODRAPE™
Continue to monitor the infant's temperature during the use of the NEODRAPE™
NEODRAPE™ is intended to be used for up to an hour after delivery or during procedures that have a duration of an hour or less.
Do not use NEODRAPE™ for longer than 24 hours.

### CONCLUSION

The exemplary embodiments of the invention were chosen and described above in order to explain the principles of the invention and its practical applications so as to enable others skilled in the art to utilize the inventions including various embodiments and various modifications as are suited to the particular uses contemplated. The examples provided herein are not intended as limitations of the present invention. For example, all of the examples refer to use of the embodiments with infants. Uses on other persons than infants may be appropriate. Moreover, the invention may have veterinary or other uses. Other embodiments will suggest themselves to those skilled in the art. Therefore, the scope of the present invention is to be limited only by the claims below.

1. A drape for use with an infant, comprising:
a sheet made of substantially biocompatible medical grade plastic,
the sheet being at least substantially sterile,
the sheet being substantially without static,
the sheet being substantially transparent,
the sheet being at least flexible enough to allow for being folded over or around the infant,
the sheet being substantially non-abrasive to skin of the infant, and
the sheet being substantially fluid-impermeable.

2. The drape of claim 1, wherein the sheet allows for heat from an external source to penetrate through the sheet to the infant.

3. The drape of claim 1, wherein the biocompatible medical grade plastic comprises polyethylene.

4. The drape of claim 1, wherein the sheet comprises at least a fenestration.

5. The drape of claim 4, wherein the sheet comprises at least a perforation from the fenestration to an edge of the sheet.

6. A drape for use with an infant, comprising:
a polyethylene plastic sterile sheet sized large enough at least to substantially cover all but the head of the infant so as to create a substantially sterile field between the infant and the sheet;
the sheet having at least a fenestration; and
the sheet having at least a perforation between the fenestration and an edge of the sheet.

7. The drape of claim 6, wherein the sheet is substantially transparent.

8. The drape of claim 6, wherein the sheet is substantially 2.0 millimeters in thickness.

9. The drape of claim 6, further comprising:
a means to secure the sheet.

10. The drape of claim 9, wherein the securing means comprises at least an adhesive strip on the sheet.

11. The drape of claim 6, wherein the sheet is substantially without static.

12. The drape of claim 6, wherein the sheet allows for heat to penetrate.

13. The drape of claim 6, wherein the sheet is flexible enough to mold to conform to body contours of the infant.

14. The drape of claim 6, wherein the sheet is flexible enough to fold around the infant.

15. The drape of claim 6, wherein the sheet is non-abrasive with respect to skin of the infant.

16. The drape of claim 6, wherein the sheet is substantially fluid-impermeable.

17. A kit for use in conserving body temperature of an infant, comprising:
a package enclosing a folded polyethylene wrap;
the wrap being sterilized in the package;
the package being openable so as to allow selective removal of the folded wrap from the package;
the wrap being selectively unfoldable to a size at least to substantially cover all but the head of the infant; and
the wrap being flexible enough to fold around the infant.

18. The kit of claim 17, wherein the wrap is substantially transparent.

19. The kit of claim 17, wherein the wrap is non-abrasive with respect to skin of the infant.

20. The kit of claim 17, wherein the wrap is substantially fluid-impermeable.
